(19) United States
(12) Patent Application Publication
(10) Pub. No.: US 2025/0261699 A1
(43) Pub. Date: Aug. 21, 2025

(54) AEROSOL PROVISION DEVICE

(71) Applicant: NICOVENTURES TRADING LIMITED, London (GB)

(72) Inventor: Darryl BAKER, London (GB)

(21) Appl. No.: 18/858,384

(22) PCT Filed: Apr. 21, 2023
(86) PCT No.: PCT/GB2023/051043

§ 371 Date: Oct. 21, 2024

(30) Foreign Application Priority Data
Apr. 22, 2022 (GB) 2205855.6

(51) Int. Cl. A24F 40/53; A24F 40/51; A24F 40/65; A61B 5/117

(52) U.S. Cl. CPC A24F 40/53; A24F 40/51; A24F 40/65; A61B 5/117

(57) ABSTRACT

There is provided an aerosol provision device, for providing an aerosol for inhalation by a user, comprising: control circuitry for controlling an activation state of the aerosol provision device; a biometric detector arranged to detect a biomarker associated with a user of the aerosol provision device and provide a signal to the control circuitry; and, a communication module for communicating with a database or computing arrangement on receipt of a signal from the control circuitry, wherein the communication module is arranged to provide a signal to the control circuitry on receipt of a signal from the database or computing arrangement, wherein the control circuitry is arranged to update an activation state of the aerosol provision device in response to receiving a signal from the communication module, and wherein the biomarker is associated with exhalate and/or saliva of a user.

### TECHNICAL FIELD

The present invention relates to an aerosol provision device, an aerosol-generating system, a method of providing an aerosol for inhalation by a user, and aerosol provision means.

### BACKGROUND

Aerosol provision systems are known. Common systems use heaters which are activated by a user to create an aerosol by an aerosol provision device from an aerosol generating material which is then inhaled by the user. The device may be activated by a user at the push of a button or merely by the act of inhalation. Modern systems can use consumable elements containing the aerosol generating material. It can be desirable for the manufacturer to enable control over the activation of the systems. This may avoid the activation of the system in undesirable circumstances.

The present invention is directed toward solving some of the above problems.

### SUMMARY

Aspects of the invention are defined in the accompanying claims.

In accordance with some embodiments described herein, there is provided an aerosol provision device, for providing an aerosol for inhalation by a user, comprising: control circuitry for controlling an activation state of the aerosol provision device; a biometric detector arranged to detect a biomarker associated with a user of the aerosol provision device and provide a signal to the control circuitry; and, a communication module for communicating with a database or computing arrangement on receipt of a signal from the control circuitry, wherein the communication module is arranged to provide a signal to the control circuitry on receipt of a signal from the database or computing arrangement, wherein the control circuitry is arranged to update an activation state of the aerosol provision device in response to receiving a signal from the communication module, and wherein the biomarker is associated with exhalate and/or saliva of a user.

Such an arrangement is able to identify a potential user of the aerosol provision device and allow authorised users to operate the device. In particular, the arrangement uses a biometric detector that utilises distinctive biomarkers to identify a user. Such an arrangement is particularly effective in detecting exhalation or saliva markers that are characteristic of a user. In particular, the device allows for high confidence recognition of users based on breath/saliva components.

The aerosol provision device of the present invention is able to operate in “offline” or “online” mode when identifying potential users. In this way, a valid user may operate the device in offline environment provided the user satisfies the criteria for operation. The user experience of the device is thereby improved.

The arrangement is also able to provide a “plug-and-play” approach. In that, the user may activate the device on first usage and the information from the biometric detector can be used to alter the activation state of the device. In this way, a valid user may have a full range of operational capabilities provided while an invalid user may have only a reduced subset or indeed no operational capabilities provided.

In accordance with some embodiments described herein, there is provided an aerosol-generating system, for providing an aerosol for inhalation by a user, comprising: an aerosol provision device comprising control circuitry for controlling an activation state of the aerosol provision device; a biometric detector arranged to detect a biomarker associated with a user of the aerosol provision device and provide a signal to the control circuitry; and, a communication module for communicating with a database or computing arrangement on receipt of a signal from the control circuitry, wherein the communication module is arranged to provide a signal to the control circuitry on receipt of a signal from the database or computing arrangement, wherein the control circuitry is arranged to update an activation state of the aerosol provision device in response to receiving a signal from the communication module, and
wherein the biomarker is associated with exhalate and/or saliva of a user.

In accordance with some embodiments described herein, there is provided a method of providing an aerosol for inhalation by a user, the method comprising: detecting, by a biometric detector, a biomarker associated with a user of an aerosol provision device; providing, by the biometric detector, a signal to control circuitry of the aerosol provision device; communicating, by a communication module, with a database or computing arrangement, on receiving a signal from the control circuitry; communicating, by a communication module, with the control circuitry, on receiving a signal from the database or computing arrangement; and, updating, by the control circuitry, an activation state of the aerosol provision device in response to receiving a signal from the communication module, wherein the biomarker is associated with exhalate and/or saliva of a user.

In accordance with some embodiments described herein, there is provided aerosol provision means, for providing an aerosol for inhalation by a user, comprising: control means for controlling an activation state of the aerosol provision means; biometric detecting means arranged to detect a biomarker associated with a user of the aerosol provision means and provide a signal to the control means; and, communication means for communicating with a database or computing arrangement on receipt of a signal from the control means, wherein the communication means is arranged to provide a signal to the control means on receipt of a signal from the database or computing arrangement, wherein the control means is arranged to update an activation state of the aerosol provision means in response to receiving a signal from the communication means, and wherein the biomarker is associated with exhalate and/or saliva of a user.

### DESCRIPTION OF DRAWINGS

The present teachings will now be described by way of example only with reference to the following figures:

FIG. 1 is a schematic view of an aerosol provision device according to an example;

FIG. 2 is a schematic view of an aerosol provision system according to an example; and,

FIG. 3 is a flow diagram according to an example.

While the invention is susceptible to various modifications and alternative forms, specific embodiments are shown by way of example in the drawings and are herein described in detail. It should be understood, however, that the drawings and detailed description of the specific embodiments are not intended to limit the invention to the particular forms disclosed. On the contrary, the invention covers all modifications, equivalents and alternatives falling within the scope of the present invention as defined by the appended claims.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to aerosol provision systems, which may also be referred to as aerosol provision systems, such as e-cigarettes. Throughout the following description the term “e-cigarette” or “electronic cigarette” may sometimes be used, but it will be appreciated this term may be used interchangeably with aerosol provision system/device and electronic aerosol provision system/device. Furthermore, and as is common in the technical field, the terms “aerosol” and “vapour”, and related terms such as “vaporise”, “volatilise” and “aerosolise”, may generally be used interchangeably.

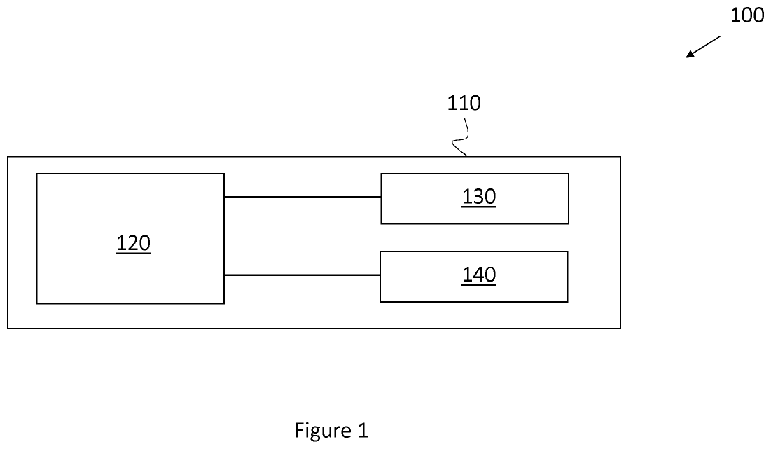

FIG. 1 illustrates a schematic view of an example of an aerosol provision device 100 according to the present invention. The aerosol provision device 100 has an aerosol provision device housing 110. The aerosol provision device 100 has control circuitry 120. The control circuitry 120 is arranged to control an activation state of the aerosol provision device 100. The aerosol provision device 100 comprises a biometric detector 130 arranged to detect a biomarker associated with a user of the aerosol provision device 100 and provide a signal to the control circuitry 120. The device 100 comprises a communication module 140 for communicating with a database or computing arrangement on receipt of a signal from the control circuitry 120, wherein the communication module 140 is arranged to provide a signal to the control circuitry 120 on receipt of a signal from the database or computing arrangement. The control circuitry 120 is arranged to update an activation state of the aerosol provision device 100 in response to receiving a signal from the communication module 140 associated with a user. The biometric detector 130 is arranged to detect a biomarker associated with exhalate and/or saliva of a user.

The biomarkers discussed herein advantageously are easily provided by a user but would be very difficult to artificially reproduce by bad users attempting to fraudulently access the device. In particular, the device can be arranged to detect one or more biomarkers associated with a user. It would be very difficult to artificially reproduce an array of biomarkers in a reasonable ratio so as to satisfy the device that an authorised user is attempting user recognition. The device may provide the signals of each biomarker to a determining module such as a database or the like, which can recognise significantly outlying ratios of biomarkers. Such recognition may increase the likelihood of recognition of fraudulent access behaviour being attempted on the device.

The biometric detector 130 is arranged to detect a biomarker and provide a signal to the control circuitry 120. The control circuitry 120 sends a subsequent signal to the communication module 140. The module 140 communicates with a database or computing arrangement to obtain an estimate on the age or identity of the user providing the biomarker signal. The module 140 relays this age or identity to the control circuity 120.

In an example, when the detected age of the user is suitable for use of the aerosol provision device 100, the user may then be allowed to access the functionality of the device 100. In another example, when the identity of the user is deemed authorised for using the aerosol provision device 100, the user may then be allowed to access the functionality of the device 100. In an example, detecting the age of the user as suitable for use but not detecting the specific identity leads to the user being allowed a reduced sub set of functionalities of the device 100. This encourages registration of the user with a device 100 to unlock full functionality. This in turn increases the accuracy of the assessment of the user against unknown users. This provides an increased robustness in the arrangement.

The signal taken from the user may be compared against a database of known users, or known values with associated age ranges.

In use therefore the biometric detector 130 detects a biomarker associated with a user and provides this to the control circuitry 120. The control circuitry 220 may provide a corresponding signal to the communication module 140 (via wireless or wired means). The communication module 140 may communicate with an on board or remote database to compare the signal provided by the user to signals previously recorded for the user (for example during a set up phase or the like, or recorded over time of usage).

This database provides a broader collection of results against which to compare the present signal. This increases the reliability of the arrangement in detecting and identifying the user and/or the age of the user. This also accounts better for gradual changes in e.g. the exhalation of a user—rather than comparing against a set of exhalations provided solely in a setup phase, which may have occurred a relatively long time prior to the present use request.

Alternatively or additionally, the signal may be sent to a remote or on board computing arrangement. The computing arrangement may utilise an artificial intelligence arrangement for assessing the user identity or user age based on the signals received from the control circuitry 120 and those previously provided for authorised users of the device 100. This may also account for discrepancies over time in use from any initial setup phase recording of data of the user.

In the above example, the communication module 140 receives signals from the control circuitry 120 and may compare the signal or signals against a database. The database may be stored in a memory on board the device 100. This memory is on board memory and the database is therefore also on board the device 100. This may be advantageous as the device 100 need not have a communications element in the device 100 to communicate with a remotely held database, and the device 100 need not be connected to a communications network to access a remotely held database prior to each use session.

In a different example, the database of user properties is held remotely, and the device 100 has the communication module 140 to contact the database. The communication module 140 may contact the database with a request for analysis of the signals from the control circuitry 120 (received in turn from the biometric detector 130). The communication module 140 is then provided with an indication as to whether the potential user is recognised as authorised (whether via identity or age range recognition) or not. Such a signal is then forwarded to the control circuitry 120. This arrangement may be advantageous as the device 100 need not include a memory element for carrying the database and the database can be remotely updated ensuring the device 100 need not have the on board database regularly updated. In this way recently obtained data on the user may provided to all relevant devices 100 as soon as the amendments are uploaded to the central database. In this way, users can be provided with the updates without each needing to update their own device 100.

In an example, the control circuitry 120 is arranged to update an activation state of the aerosol provision device 100 to a non-operating state in response to receiving signals from the communication module 140 associated with a non-authorised user (whether via identity or age range estimation). In an example, the control circuitry 120 is arranged to update an activation state of the aerosol provision device 100 to an operating state in response to receiving signals from the communication module 140 associated with an authorised user.

The communication module 140 may be arranged to use wireless communication to communicate with any of the control circuitry 120, the on board or remote database or the on board or remote computing arrangement. The communication module 140 is arranged to use at least one of Bluetooth™, Bluetooth Low Energy™, ZigBee™, WiFi™, Wifi Direct™, GSM, 2G, 3G, 4G, 5G, LTE, NFC, or RFID.

The biometric detector 130 detects aspects of exhalate or saliva and provides this for user recognition and/or age estimation to the control circuitry 120. Recognition of a user may involve comparing the present signals to those known as being characteristic of a specific user. This then involves checking whether that specific user is authorised to use the device 100. Age estimation of the user involves obtaining the signal and comparing that signal to known age-related biomarkers. In an example, the biometric detector 130 is arranged to detect concentrations of at least one of: ATP; citrulline; creatinine; and, glutathione disulphide. These compounds have a strong correlation between concentration and age. The concentration of each drops with age, apart from ATP which has been shown to increase. As such, each of these (which may be detected by the detector 130) can be used by a computing arrangement to provide an estimate of the age range of the user.

In another example, the biometric detector 130 is arranged to detect a temperature of exhalate of a user.

In an example, the biometric detector 130 is located in a mouthpiece of the aerosol provision device 100 or at a portion of a housing 110 that contacts a user's mouth or lips during use. This increases the reliability with which compounds in exhalate or saliva can be received by the biometric detector 130. The closer to the source, the more reliable the supply of compound for analysis.

The aerosol provision device 100 may comprise heating arrangements or the like for providing an aerosol from a consumable—the consumable may contain some aerosol generating material or the like. The control circuitry 120 may control the heating arrangement (or the like) according to the signal received from the biometric detector 130. The detector 130 enables the control circuitry 120 to ascertain whether the user is a valid user of the aerosol provision device 100. In an example, the biometric detector 130 may provide a signal to the control circuitry 120, the control circuitry 120 may recognise the signal as belonging to a valid user and enable any operational mode of the device 100. In another example, the biometric detector 130 may provide a signal to the control circuitry 120, the control circuitry 120 may recognise the signal as belonging to an invalid user and prevent any operational mode of the device 100. In this way, protection over the use of the aerosol provision device 100 is provided. This protection is not user intensive; the protection does not rely on the user taking several actions to provide the protection, rather the protection is provided for the user via the biometric detector 130, increasing the user experience of the device 100.

The present invention involves updating or changing an activation state of the aerosol provision device. In an “operating state”, elements of the aerosol provision device used to generate an aerosol (such as an atomiser, heater or the like) may be activated. The specific activation of the device may require an additional input which may be inhalation on the device (or a specific mouthpiece of the device), pressing a button on the device or the like. Alternatively, the device may automatically generate aerosol by a heater in response to receiving a signal associated with an authorised or valid user from the biometric detector. The control circuitry may receive such a signal from the biometric detector and send a signal to the heater arrangement or the like to provide an aerosol from an aerosol generating material that may contained within, or separate to, the aerosol provision device.

The term “operating state” refers to a state in which the device may be used to provide an aerosol. The device can provide a large number of “operating states”. Specific aerosol generating materials or aerosol generating material consumables may provide a suitable aerosol for a user under a suitable heating profile (fast, slow, high temperature, low temperature, etc.) or via a suitable heating mechanism (heater element, atomiser, vibrating plate, etc.). As such specific, operating states may dictate the performance of the system, such as selecting the heating mechanisms used and the heating profile used based on the identified user—one user may be able to store a preferred heating profile to be associated with their biometric data. In this way, in response to identification of user 1, corresponding heating mechanism 1 is offered to provide heating profile 1 on the aerosol generating material. In response to identification of user 2, corresponding heating mechanism 2 may be offered to provide heating profile 2 on the aerosol generating material. In this way, different users are provided a bespoke aerosol, in response to recognition of that user. This improves the ease of use, and the use experience, of a device for a user.

In an example, the biometric detector 130 is arranged to detect at least one of: exhalate pressure; exhalate speed; exhalate volume; exhalate chemical composition; time period between exhalations. Such a multi-sensor arrangement, or multi-characteristic detection, provides resilience against one inaccurate reading.

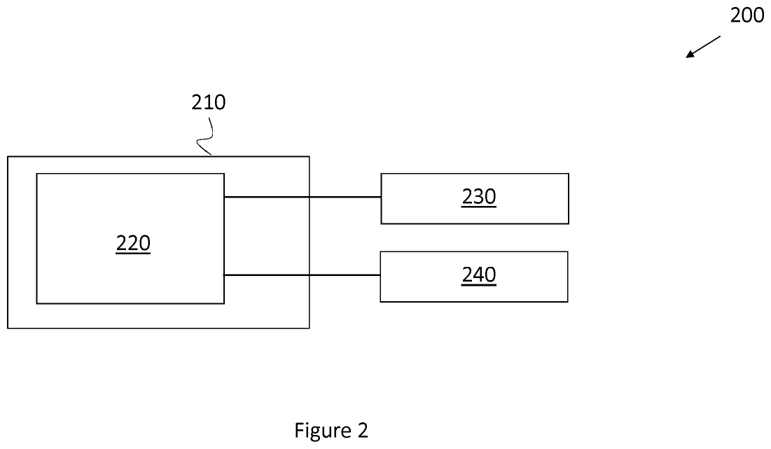

Referring now to FIG. 2, there is a shown a similar system 200 to the device 100 of FIG. 1. Similar features, to those features used in FIG. 1, are shown with the reference numerals increased by 100. For example, the device 100 of FIG. 1 is similar to the system 200 of FIG. 2. Similar or identical features may not be discussed for conciseness.

The system 200 of FIG. 2 has an aerosol provision device 210 comprising control circuitry 220. The control circuitry 220 is connected (wirelessly or wired) to a biometric detector 230. The system has a communication module 240 for communicating with a database or computing arrangement or the like, as explained above.

The example shown in FIG. 2 shows biometric detector 230 and communication module 240 as not integral with the aerosol provision device 210. In an example, at least one is not integral with the aerosol provision device 210. This enables the manufacturing of the aerosol provision device 310 to be simpler and therefore cheaper. The biometric detector 230 may be carried by a user alongside the aerosol provision device 210 and, prior to use of the aerosol provision device 110, the user may utilise the biometric detector 230, satisfy user authorisation, and then use the aerosol provision device 210. The aerosol provision device 210 may be in a default operating state of non-operation, i.e. the device 210 cannot be used without first satisfying user authentication by the biometric detector 230. This prioritises safety and ensures that unauthorised users are unable to activate the device 210. The biometric detector 230 may be arranged to communicate with the control circuitry 220 over a short distance communication system such as Bluetooth TM or Infrared or RFID. This ensures that the user near the biometric detector 230 also is near the aerosol provision device 210 and hinders one user accidentally satisfying authorisation with the biometric detector 230 while the aerosol provision device 210 is in the possession of a second unauthorised user.

The biometric detector 230 or communications module 240 may be a mobile device or smart device belonging to the user of the aerosol provision device 210, with capability of receiving and processing exhalate or saliva from the user. This arrangement utilises the operability of a separate component thereby reducing the manufacturing requirements on the aerosol provision device 210. The smart device may be linked to the aerosol provision device 210 via an app or the like.

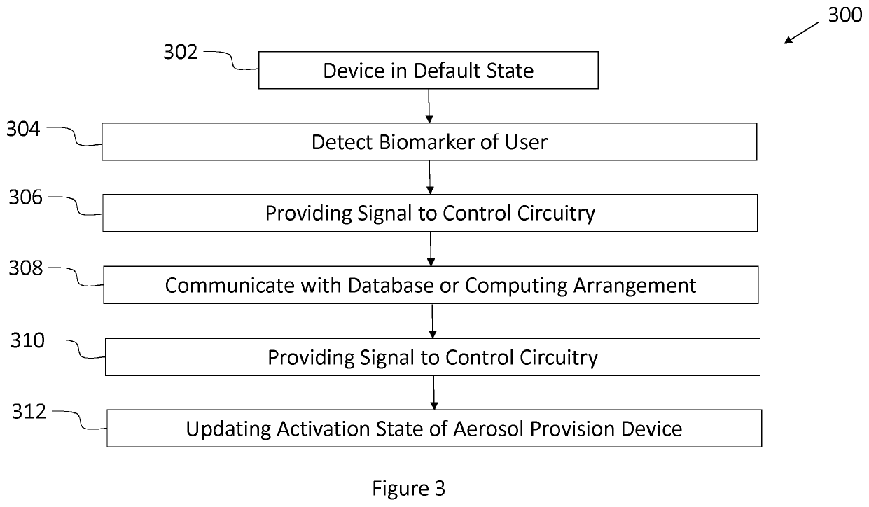

FIG. 3 shows a method 300 of use of an aerosol provision device. In the method 300, the device may start in a default state 302, which may be a non-operating state such that non-valid users cannot use the device. Alternatively, the default operating state may be a restricted operating state where only partial operation of the device is possible.

When a user intends to use the aerosol provision device, the device or system detects a biomarker of the user 304. The biomarker is detected using a biometric detector (which may contain a saliva/exhalate sensor alongside a number of individual sensors/detectors as explained above). The detector uses the sensor to detect physical or chemical characteristics of the exhalate and/or saliva. The signal from the biometric detector is provided to control circuitry of the device 306. In step 308, the device or system communicates with a database or computing arrangement. This involves comparison or processing of the signal to identify the user or to estimate the age of the user. This may involve use of a database for comparison or an artificial intelligence arrangement to estimate age. In step 310, the signal from the database or computing arrangement is provided to the control circuitry. In step 312, there is an update of the activation state of the aerosol provision device.

This may take the form of updating the activation state to an operating state such as fully enabled for use, partially enabled for use or prevented from use. As discussed above, this change is based on the detected biomarker (or biomarkers) from the user. These biomarkers are used to ascertain a suitability of use for the user. For example, a first authorised user (the owner of the device) may have all functionality unlocked when identified by the biometric detector while a second authorised user (e.g. a friend of the owner borrowing the device) may have only a limited functionality unlocked by having an age range estimated as suitable for use with the device.

This method provides a user-friendly verification process that provides suitable protection against invalid users while not impeding use for valid users. The method offers a balance between overly strict and overly lenient access protection for the device-particularly with flexibility provided by the default activation state that can be selected by a user to be more strict, less user friendly or more lenient, more user friendly.

The technique is less impactful on a user when in the more lenient, more user friendly arrangement wherein the default state is active. The user may be recognised during use and the device does not therefore impede the user's use of the device significantly. Such a method is extremely user friendly.

The method and device disclosed herein enable protection over the use of the device without requiring an arduous authorisation process. This improves the user experience of the device and the safety of general use of the device.

The term “in response to” is used herein to indicate a second event (such as a signal or change of state of an aerosol provision device) that occurs subsequent to a first event. The second event may occur at a later time, after a predetermined time, or immediately after the first event.

The device and system herein are described as comprising several components that enable several advantages. The components may be disclosed as on-board the device or within the system. The components may be distributed and therefore not necessarily be located on-board the device. The functionality of the device can be provided by communicatively connected components, and such communication may be wireless, enabling such distribution. At which point it is reasonable to foresee that a distributed array of components will operate in the manner of the devices and systems disclosed herein. Components of the device or system may be contained in a further device such as a smartphone, computer, or remote server or the like.

In a particular example, the device disclosed herein may operate with a flavour pod which is replaceable in the device—this may be referred to as a consumable. The flavour may be any of tobacco and glycol and may include extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamon, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof.

When combined with an aerosol generating medium, the aerosol provision device as disclosed herein may be referred to as an aerosol provision system.

Thus there has been described an aerosol provision device, for providing an aerosol for inhalation by a user, comprising: control circuitry for controlling an activation state of the aerosol provision device; a biometric detector arranged to detect a biomarker associated with a user of the aerosol provision device and provide a signal to the control circuitry; and, a communication module for communicating with a database or computing arrangement on receipt of a signal from the control circuitry, wherein the communication module is arranged to provide a signal to the control circuitry on receipt of a signal from the database or computing arrangement, wherein the control circuitry is arranged to update an activation state of the aerosol provision device in response to receiving a signal from the communication module, and wherein the biomarker is associated with exhalate and/or saliva of a user.

The aerosol provision system may be used in a tobacco industry product, for example a non-combustible aerosol provision system.

In one embodiment, the tobacco industry product comprises one or more components of a non-combustible aerosol provision system, such as a heater and an aerosolizable substrate.

In one embodiment, the aerosol provision system is an electronic cigarette also known as a vaping device.

In one embodiment the electronic cigarette comprises a heater, a power supply capable of supplying power to the heater, an aerosolizable substrate such as a liquid or gel, a housing and optionally a mouthpiece.

In one embodiment the aerosolizable substrate is contained in or on a substrate container. In one embodiment the substrate container is combined with or comprises the heater.

In one embodiment, the tobacco industry product is a heating product which releases one or more compounds by heating, but not burning, a substrate material. The substrate material is an aerosolizable material which may be for example tobacco or other non-tobacco products, which may or may not contain nicotine. In one embodiment, the heating device product is a tobacco heating product.

In one embodiment, the heating product is an electronic device.

In one embodiment, the tobacco heating product comprises a heater, a power supply capable of supplying power to the heater, an aerosolizable substrate such as a solid or gel material.

In one embodiment the heating product is a non-electronic article.

In one embodiment the heating product comprises an aerosolizable substrate such as a solid or gel material, and a heat source which is capable of supplying heat energy to the aerosolizable substrate without any electronic means, such as by burning a combustion material, such as charcoal.

In one embodiment the heating product also comprises a filter capable of filtering the aerosol generated by heating the aerosolizable substrate.

In some embodiments the aerosolizable substrate material may comprise an aerosol or aerosol generating agent or a humectant, such as glycerol, propylene glycol, triacetin or diethylene glycol.

In one embodiment, the tobacco industry product is a hybrid system to generate aerosol by heating, but not burning, a combination of substrate materials. The substrate materials may comprise for example solid, liquid or gel which may or may not contain nicotine. In one embodiment, the hybrid system comprises a liquid or gel substrate and a solid substrate. The solid substrate may be for example tobacco or other non-tobacco products, which may or may not contain nicotine. In one embodiment, the hybrid system comprises a liquid or gel substrate and tobacco.

In order to address various issues and advance the art, the entirety of this disclosure shows by way of illustration various embodiments in which the claimed invention(s) may be practiced and provide for a superior electronic aerosol provision system. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future.

1. An aerosol provision device, for providing an aerosol for inhalation by a user, comprising:
control circuitry for controlling an activation state of the aerosol provision device;
a biometric detector arranged to detect a biomarker associated with a user of the aerosol provision device and provide a signal to the control circuitry; and,
a communication module for communicating with a database or computing arrangement on receipt of a signal from the control circuitry, wherein the communication module is arranged to provide a signal to the control circuitry on receipt of a signal from the database or computing arrangement,
wherein the control circuitry is arranged to update an activation state of the aerosol provision device in response to receiving a signal from the communication module, and
wherein the biomarker is associated with exhalate and/or saliva of a user.

2. An aerosol provision device according to claim 1, wherein the control circuitry is arranged to update an activation state of the aerosol provision device to a non-operating state in response to receiving signals from the communication module associated with a non-authorised user.

3. An aerosol provision device according to claim 1, wherein the control circuitry is arranged to update an activation state of the aerosol provision device to an operating state in response to receiving signals from the communication module associated with an authorised user.

4. An aerosol provision device according to 1, wherein the communication module is arranged to communicate with at least one of: a remote database; a remote computing arrangement; an onboard database; and, an onboard computing arrangement.

5. An aerosol provision device according to claim 1, wherein the communication module is arranged to use wireless communication.

6. An aerosol provision device according to claim 1, wherein the communication module is arranged to use at least one of Bluetooth™, Bluetooth Low Energy™, ZigBee™, WiFi™, Wifi Direct™, GSM, 2G, 3G, 4G, 5G, LTE, NFC, or RFID.

7. An aerosol provision device according to claim 1, wherein the biometric detector is arranged to detect concentrations of at least one of: ATP; citrulline; creatinine; and, glutathione disulphide.

8. An aerosol provision device according to claim 1, wherein the biometric detector is located in a mouthpiece of the aerosol provision device or at a portion of a housing that contacts a user's mouth or lips during use.

9. An aerosol provision device according claim 1, wherein the biometric detector is arranged to detect a temperature of exhalate of a user.

10. An aerosol-generating system, for providing an aerosol for inhalation by a user, comprising:
an aerosol provision device comprising control circuitry for controlling an activation state of the aerosol provision device;
a biometric detector arranged to detect a biomarker associated with a user of the aerosol provision device and provide a signal to the control circuitry; and,
a communication module for communicating with a database or computing arrangement on receipt of a signal from the control circuitry, wherein the communication module is arranged to provide a signal to the control circuitry on receipt of a signal from the database or computing arrangement,
wherein the control circuitry is arranged to update an activation state of the aerosol provision device in response to receiving a signal from the communication module, and
wherein the biomarker is associated with exhalate and/or saliva of a user.

11. An aerosol-generating system according to claim 10, wherein at least one of the biometric detector and the communication module is not integral with the aerosol provision device.

12. A method of providing an aerosol for inhalation by a user, the method comprising:
detecting, by a biometric detector, a biomarker associated with a user of an aerosol provision device;
providing, by the biometric detector, a signal to control circuitry of the aerosol provision device;
communicating, by a communication module, with a database or computing arrangement, on receiving a signal from the control circuitry;
communicating, by a communication module, with the control circuitry, on receiving a signal from the database or computing arrangement; and,
updating, by the control circuitry, an activation state of the aerosol provision device in response to receiving a signal from the communication module,
wherein the biomarker is associated with exhalate and/or saliva of a user.

13. The method of claim 12, wherein updating, by the control circuitry, an activation state of the aerosol provision device comprises updating an activation state of the aerosol provision device to a non-operating state in response to receiving signals from the biometric detector and the material detector associated with a non-authorised user.

14. The method of claim 12, further comprising updating an activation state of the aerosol provision device to an operating state in response to receiving signals from the biometric detector and the material detector associated with an authorised user.

15. The method of claim 12, wherein communicating, by a communication module, with a database or computing arrangement comprises:
communicating, by a communication module, with at least one of a remote database; a remote computing arrangement; an onboard database; and, an onboard computing arrangement.

16. The method of claim 12, wherein communicating, by a communication module, with a database or computing arrangement comprises wirelessly communicating, by a communication module, with a database or computing arrangement.

17. The method of claim 12, wherein detecting, by a biometric detector, a biomarker associated with a user of an aerosol provision device comprises:
detecting concentrations of at least one of: ATP; citrulline; creatinine; and, glutathione disulphide in exhalate and/or saliva of a user.

18. The method of claim 12, wherein detecting, by a biometric detector, a biomarker associated with a user of an aerosol provision device comprises:
detecting temperature of exhalate of a user.

19. (canceled)
